(19) United States
(12) Patent Application Publication
(10) Pub. No.: US 2012/0078340 A1
(43) Pub. Date: Mar. 29, 2012

(54) STENT POSITIONING AND DEPLOYMENT ASSEMBLY AND METHOD FOR TREATING A SIDE-BRANCH VESSEL

(76) Inventor: Manish P. Gupta, Cherry Hill, NJ (US)

(21) Appl. No.: 12/893,730

(22) Filed: Sep. 29, 2010

(51) Int. Cl. A61F 2/84

(52) U.S. Cl. 623/1.11

(57) ABSTRACT

A stent positioning and deployment assembly for a constricted branched vessel is provided which includes a guide wire, a stent, a first inflatable balloon disposed within the stent and being separable from the stent, and a second inflatable balloon located proximate the first balloon. The first and second balloons each have a collapsed state and an expanded state. The inflation of the first and second balloons are controllable independently of each other, such that each of the first and second balloons may be placed into the expanded state independently of each other.

### BACKGROUND OF THE INVENTION

Surgical stents are typically implanted in body lumens, such as vessels and arteries, to support a region of the lumen adjacent a lesion or build-up of plaque. Surgical stents are generally cylindrical in form. Prior to use and during implantation, the stent is in a generally collapsed form. After implantation and deployment, the stent is in a generally expanded or inflated form.

In their collapsed forms, stents are typically fixed on or over an inflatable balloon. The balloon and stent assembly, with both the balloon and stent in their collapsed forms, is guided into the body lumen over a wire which is positioned within a support structure, such as a catheter, which is controlled by the surgeon. When the balloon and stent assembly is situated at the desired position within the body lumen, the balloon is deployed, causing the balloon to expand radially or inflate which, in turn, causes the stent to expand radially. When the stent is expanded to the desired degree (i.e., to the radial size of interior of the body lumen), the balloon can then be deflated and removed with the catheter. The stent, however, remains in place in the body lumen to support the body lumen.

However, prior art stent assemblies suffer from deficiencies, particularly when such conventional assemblies are used in branched vessels, such as coronary arteries. Branched vessels generally have tortuous paths and are curving bodies, and it often proves difficult to position prior art balloon and stent assemblies at the desired site without damaging the main vessel of the branched vessel.

### BRIEF SUMMARY OF THE INVENTION

Briefly stated, the present invention is directed to a stent positioning and deployment assembly for a constricted branched vessel. The assembly includes a guide wire, a stent, a first inflatable balloon disposed within the stent and which is separable from the stent, and a second inflatable balloon which located proximate the first balloon. The first and second balloons each have a collapsed state and an expanded state. Also, the inflation of the first and second balloons are controllable independently of each other, such that each of the first and second balloons may be placed into the expanded state independently of each other.

In another embodiment, the present invention is directed to a method of positioning and implanting a stent in a branched vessel including a main vessel and at least one constricted side branch vessel. The method comprises the steps of (i) guiding an assembly through the main vessel toward the side branch vessel, the assembly including a guide wire, a stent, a first balloon disposed within the stent and being separable from the stent, a second balloon located proximate the first balloon, a first fluid source coupled to the first balloon, and a second fluid source coupled to the second balloon; (ii) positioning the assembly in the branched vessel such that the first balloon and stent are located within the constricted side branch vessel at the area of the constriction, the first balloon and the stent being in a collapsed state, and the second balloon is located at the point where the constricted side branch vessel branches off from the main vessel, the second balloon being in a collapsed state; (iii) injecting fluid from the second fluid source into the second balloon to inflate the second balloon; (iv) injecting fluid from the first fluid source into the first balloon to inflate the first balloon and to place the stent into an expanded state, the expanded stent causing the constricted vessel to expand; (v) removing the injected fluid from the first and second balloons to place the first and second balloons in collapsed states; and (vi) withdrawing the guide wire and first and second balloons from the branched vessel.

### BRIEF DESCRIPTION OF THE DRAWINGS

The foregoing summary, as well as the following detailed description of the invention, will be better understood when read in conjunction with the appended drawings. For the purpose of illustrating the invention, there is shown in the drawings an embodiment which is presently preferred. It should be understood, however, that the invention is not limited to the precise arrangements and instrumentalities shown. In the drawings:

FIG. 1 is a right side schematic elevation view of a stent positioning and deployment assembly in accordance with a preferred embodiment of the present invention; and

FIG. 2 is a right side schematic elevation view of the stent positioning and deployment assembly shown in FIG. 1 positioned and deployed within a branched vessel.

### DETAILED DESCRIPTION OF THE INVENTION

Certain terminology is used in the following description for convenience only and is not limiting. The words “right,” “left,” “lower,” and “upper” designate directions in the drawings to which reference is made. The words “inwardly” or “distally” and “outwardly” or “proximally” refer to directions toward and away from, respectively, the geometric center or orientation of the stent positioning assembly and related parts thereof. The terminology includes the above-listed words, derivatives thereof and words of similar import.

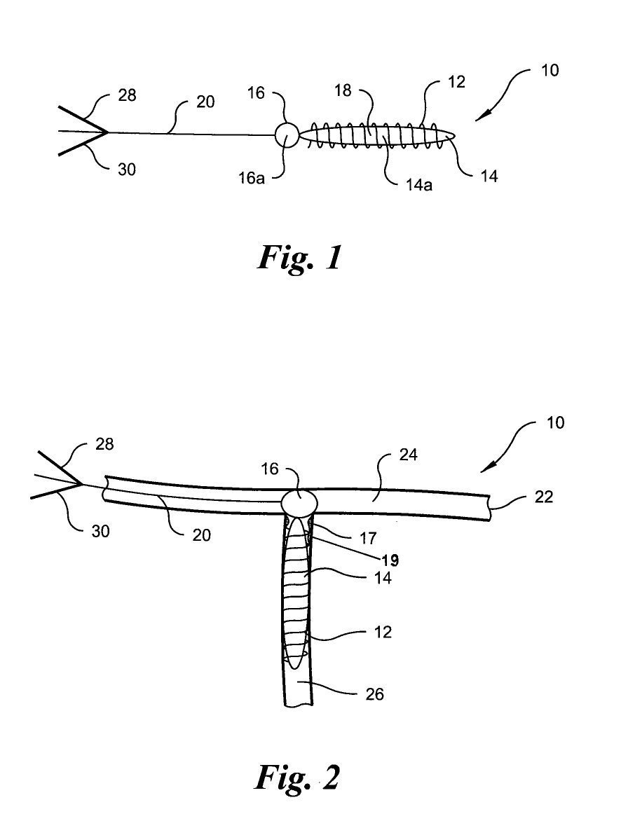

Referring to the drawings in detail, wherein like numerals indicate like elements throughout, there is shown in FIGS. 1-2, a preferred embodiment of a stent positioning assembly, generally indicated 10, for positioning, delivering and deploying a stent in a constricted branched vessel in accordance with the present invention. Specifically, the assembly 10 includes an expandable stent 12, a first balloon 14 and a second balloon 16. The assembly 10 is particularly designed for use in a branched vessel 22 including a main vessel 24 and at least one constricted side branch vessel 26. A constricted side branch vessel 26 is a side branch vessel that has a build-up of plaque deposits 17 in a specific area, such as the ostium 19 of the side branch vessel 26, or some other type of constriction. In the area of the first and second balloons 14, 16, the assembly has a certain degree of flexibility, such that the assembly may be guided into and through tortuous paths and the side branch vessel 26, where the stent 12 is to be implanted

Referring to FIG. 1, the first balloon 14 is positioned or disposed within the interior 18 of the stent 12, which has a generally coiled shape. The first balloon 14, however, remains separable from the stent 12. A guide wire 20 is also provided to guide and carry the stent 12 and first and second balloons 14, 16. It will be understood by those skilled in the art that while the guiding member is described herein as a guide wire, the guiding member may take any appropriate form. The first balloon 14 has an elongated shape and extends generally longitudinally along a portion of the guide wire 20. The second balloon 16 preferably has a generally spherical shape and is preferably positioned proximate the proximal end of the first balloon 14. Preferably, the second balloon 16 is attached to the first balloon 14 for better and more accurate positioning of the stent 12. The first and second balloons 14, 16 are preferably angioplasty balloons.

The first and second balloons 14, 16 are each inflatable or expandable, such that each balloon 14, 16 has a first collapsed state and a second expanded or inflated state. The first and second balloons 14, 16 are each essentially a hollow enclosure surrounding a respective interior 14a, 16a which can be selectively filled with and removed of a fluid under pressure. Preferably, the inflation and deflation of the first balloon 14 may be controlled independently and separately from the second balloon 16. More particularly, the first balloon 14 is operatively coupled to a first source 28 of fluid for inflation of the first balloon 14 and the second balloon 16 is operatively coupled to a second source 30 of fluid for inflation of the second balloon 16. Accordingly, each of the first and second balloons 14, 16 may be placed into the inflated or expanded state and into a deflated state independently of each other. The inflation fluid is preferably a biocompatible fluid, such as a water and saline and contrast solution. When the respective interiors of the first and second balloons 14, 16 are at least partially filled with the fluid, the first and second balloons 14, 16 expand from their collapsed states into their inflated states. The first and second fluid sources 28, 30 may be, for example, fluid supply tubes, such as catheters, coupled to the first and second balloon 14, 16 respectively.

The first balloon 14, in particular, is configured to be radially expandable. Expansion or inflation of the first balloon 14 also generally simultaneously causes the stent 12, which is compressed against the outer surface of the first balloon 14 in the collapsed configuration, to similarly radially expand. However, when the fluid is withdrawn from the first balloon 14, so as to deflate the first balloon 14 or place the first balloon 14 into the collapsed state, the stent 12 does not collapse. Instead, the stent 12 retains its expanded shape.

In use, the assembly 10 is particularly beneficial for treating the ostial or proximal part of constricted branched vessels. Initially, the assembly 10 is guided through the main vessel 24 toward the side branch vessel 26, with the stent 12 and the first and second balloons 14, 16 being in the collapsed configuration. The first balloon 14 is inserted into the branched vessel 22 prior to the second balloon 16. Specifically, the assembly 10 is advanced axially into the branched vessel 22 by the guide wire 20 until the first balloon 14 and the stent 12, in the collapsed states, are located within the constricted side branch vessel 26 at the area of the constriction 17, the ostium 19, and the second balloon 16, also in the collapsed state, is located the mouth of the side branch vessel 26. That is, the second balloon 16 should be located at the point where the constricted side branch vessel 26 branches off from the main vessel 24. When the assembly 10 is properly positioned, as shown in FIG. 2, such that the stent 12 is located at the constricted area, pressurized fluid is injected from the second fluid source 30 into the interior 16a of the second balloon 16 to inflate the second balloon 16. Once the second balloon 16 is properly positioned and inflated, as shown in FIG. 2, the main vessel 24 is effectively isolated from any activity occurring in the side branched vessel 26. Accordingly, the main vessel 24 is secure from any trauma that may be caused by deployment of the stent 12.

Next, pressurized fluid is injected from the first fluid source 28 into the interior 14a of the first balloon 14 to inflate the first balloon 14 and to cause the stent 12 to be radially expanded. The expanded stent 12 presses against the constricted side branch vessel 26, thereby causing the constricted side branch vessel 26 to also expand. The injected fluid may then be removed from the first and second balloons 14, 16 to deflate the first and second balloons 14, 16, so that the first balloon 14 is no longer exerting radial pressure against the stent 12 and the first and second balloons 14, 16 are in collapsed states. The assembly 10, including the guide wire 18 and the collapsed first and second balloons 14, 16, can then be withdrawn from the branched vessel 22. The stent 12, however, remains in place to support and maintain the expanded side branch vessel 26.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

I claim:

1. A stent positioning and deployment assembly for a constricted branched vessel comprising a guide wire, a stent, a first inflatable balloon disposed within the stent and being separable from the stent, and a second inflatable balloon located proximate the first balloon, wherein the first and second balloons each have a collapsed state and an expanded state, the inflation of the first and second balloons being controllable independently of each other such that each of the first and second balloons may be placed into the expanded state independently of each other.

2. The stent positioning and deployment assembly of claim 1, wherein the second balloon is attached to the first balloon.

3. The stent positioning and deployment assembly of claim 1, wherein the first balloon has a generally elongated shape and extends longitudinally along at least a portion of the length of the guide wire.

4. The stent positioning and deployment assembly of claim 1, wherein the second balloon has a generally spherical shape.

5. The stent positioning and deployment assembly of claim 1 further comprising a first source of fluid operably coupled to the first balloon and a second source of fluid operably coupled to the second balloon.

6. A method of positioning and implanting a stent in a branched vessel including a main vessel and at least one constricted side branch vessel, the method comprising the steps of:
(i) guiding an assembly through the main vessel toward the side branch vessel, the assembly including a guide wire, a stent, a first balloon disposed within the stent and being separable from the stent, a second balloon located proximate the first balloon, a first fluid source coupled to the first balloon, and a second fluid source coupled to the second balloon;
(ii) positioning the assembly in the branched vessel such that the first balloon and stent are located within the constricted side branch vessel at the area of the constriction, the first balloon and the stent being in a collapsed state, and the second balloon is located at the point where the constricted side branch vessel branches off from the main vessel, the second balloon being in a collapsed state;
(iii) injecting fluid from the second fluid source into the second balloon to inflate the second balloon and effectively isolate the main vessel from the constricted side branch vessel;
(iv) injecting fluid from the first fluid source into the first balloon to inflate the first balloon and to place the stent into an expanded state, the expanded stent causing the constricted vessel to expand;
(v) removing the injected fluid from the first and second balloons to place the first and second balloons in collapsed states; and
(vi) withdrawing the guide wire and first and second balloons from the branched vessel.
